(19) United States
(12) Patent Application Publication
(10) Pub. No.: US 2005/0020506 A1
(43) Pub. Date: Jan. 27, 2005

(54) Crosslinked compositions comprising collagen and demineralized bone matrix, methods of making and methods of use

(76) Inventors: Susan J. Drapeau, Cordova, TN (US); Mark Torrianni, San Juan Capistrano, CA (US); Frank Everaerts, Maastricht (NL); William F. McKay, Memphis, TN (US)

Correspondence Address: Charles R. Reeves;Woodard, Emhardt, Moriarty, McNett & Henry LLP, Bank One Center/Tower, 111 Monument Circle, Suite 3700, Indianapolis, IN, 46204-5137, US

(21) Appl. No.: 10/626,571

(22) Filed: Jul. 25, 2003

(51) Int. Cl. A61K 38/39; A61K 38/18

(52) U.S. Cl. 514/21; 514/12

(57) ABSTRACT

A composition comprising a collagen protein and demineralized bone matrix is described wherein the composition is chemically cross-linked with a carbodiimide such as N-(3-dimethylaminopropyl)-N-ethylcarbodiimide hydrochloride (EDC). The crosslinking reaction can be conducted in the presence of N-hydroxysuccinimide (NHS). The collagen can be in a porous matrix or scaffolding. The DBM can be in the form of particles dispersed in the collagen. A method of making the composition is also described wherein a collagen slurry is cast into the desired shape, freeze dried to form a porous scaffolding and infitrated with a solution comprising the cross-linking agent. The composition can be used as an implant for tissue (e.g., soft tissue or bone) engineering.

### BACKGROUND

### Technical Field

The present application relates generally to bioprosthetic devices and, in particular, to chemically cross-linked collagen based carriers comprising demineralized bone matrix (DBM) and to the use of these materials as implants such as, for example, osteoinductive implants.

### Background of the Technology

Various materials have been used to repair or regenerate bone or soft tissue that has been lost due to either trauma or disease. Typically, implantable bone repair materials provided a porous matrix (i.e., scaffolding) for the migration, proliferation and subsequent differentiation of cells responsible for osteogenesis. While the compositions provided by this approach provided a stable structure for invasive bone growth they did not promote bone cell proliferation or bone regeneration.

Subsequent approaches have used bone repair matrices containing bioactive proteins which when implanted into the bone defect provided not only a scaffolding for invasive bone ingrowth, but active induction of bone cell replication and differentiation. In general these osteoinductive compositions are comprised of a matrix which provides the scaffolding for invasive growth of the bone and anchorage dependent cells and an osteoinductive protein source. The matrix may be selected from a variety of materials including collagen, polylactic acid or an inorganic material such as a biodegradable porous ceramic. Two specific substances that have been found to induce the formation of new bone through the process of osteogenesis include demineralized bone particles or powder and bone morphogenetic proteins (BMPs).

While a wide variety of compositions have been used for tissue engineering, there still exists a need for improvements or enhancements which would accelerate and enhance bone and soft tissue repair and regeneration thereby allowing for a faster recovery and a better result for a patient receiving the implant.

### SUMMARY OF THE INVENTION

According to a first aspect of the invention, a composition is provided comprising demineralized bone matrix (DBM) and a collagen protein wherein the composition is crosslinked. The composition can be chemically crosslinked with a carbodiimide. The carbodiimide can be N-(3-dimethylaminopropyl)-N-ethylcarbodiimide hydrochloride (EDC). The composition can be chemically cross-linked with a carbodiimide in the presence of N-hydroxysuccinimide (NHS). The composition can further include one or more growth factors. The collagen protein can be in a porous scaffolding. The DBM can be in the form of particles. For example, the composition can comprise particles of DBM dispersed in a porous scaffolding comprising the collagen protein. The DBM particles can have an average particle size of up to 5 mm. For example, the DBM particles have an average particle size ranging from 53 to 850 μm.

According to a second aspect of the invention, a method of making a composition comprising a collagen protein and demineralized bone matrix is provided comprising crosslinking the composition. The composition can be chemically crosslinked with a carbodiimide. The carbodiimide can be N-(3-dimethylaminopropyl)-N-ethylcarbodiimide hydrochloride (EDC). The composition can be chemically crosslinked with a carbodiimide in the presence of N-hydroxysuccinimide (NHS). When NHS is used, the NHS can be present at an EDC/NHS ratio of 1:2 to 2:5. For example, the NHS can be present at an EDC/NHS ratio of 1:2, 2:3 or 2:5. The reaction may or may not take place in an environment with a controlled pH such as a buffer solution. The method according to this aspect of the invention can further comprise dispersing demineralized bone particles in a collagen slurry, casting the slurry into the cavity of a mold and freeze drying the cast slurry to form a porous collagen scaffolding comprising particles of the demineralized bone matrix. The slurry can, for example, be an aqueous slurry comprising the collagen protein and the DBM particles. The slurry can be at an acidic pH. According to this aspect of the invention, crosslinking can comprise infiltrating a carbodiimide crosslinking agent into pores of the porous collagen scaffolding and allowing the carbodiimide cross-linking agent to react with molecules of the collagen protein to form cross-links.

According to a third aspect of the invention, a method of treatment is provided comprising implanting into a mammal a composition comprising demineralized bone matrix (DBM) and a collagen protein wherein the composition is cross-linked. The composition can be chemically crosslinked with a carbodiimide. The composition can be used in an orthopaedic application. For example, the composition can be implanted into the spine of the mammal or into an intervertebral space of the mammal. The mammal can be a human.

According to a fourth aspect of the invention, a composition comprising demineralized bone matrix (DBM) and a collagen protein is provided wherein the composition is cross-linked via an amide linkage. The composition can comprise particles of the DBM dispersed in the collagen protein. The collagen protein can be in a porous scaffolding. The DBM particles can have an average particle size of up to 5 mm. For example, the DBM particles can have an average particle size ranging from 53 to 850 μm.

### BRIEF DESCRIPTION OF THE DRAWINGS

FIG. 1 illustrates the formation of an amide crosslinked protein matrix using a carbodiimide crosslinking agent according to the invention.

FIGS. 2-7 are images of histological sections of collagen/DBM sponges which have been implanted into rats.

### DETAILED DESCRIPTION

According to one embodiment of the invention, a composition comprising DBM in a collagen carrier is provided which provides an osteoconductive matrix for cell migration and which has an extended duration after implantation in a patient. According to further embodiment of the invention, a chemical crosslinking method is provided to crosslink a composition comprising collagen and DBM. During crosslinking, collagen molecules can be crosslinked together through reactive groups present on the collagen molecules. Also during crosslinking, collagen molecules can be crosslinked to the DBM due to the presence of reactive surface groups on the DBM. As a result, an osteoconductive matrix that lasts longer after implantation and that can still be turned over in vivo as bone is formed is provided. This method also allows control of the amount of DBM added to the matrix and optimization of the material handling characteristics of the resulting composition.

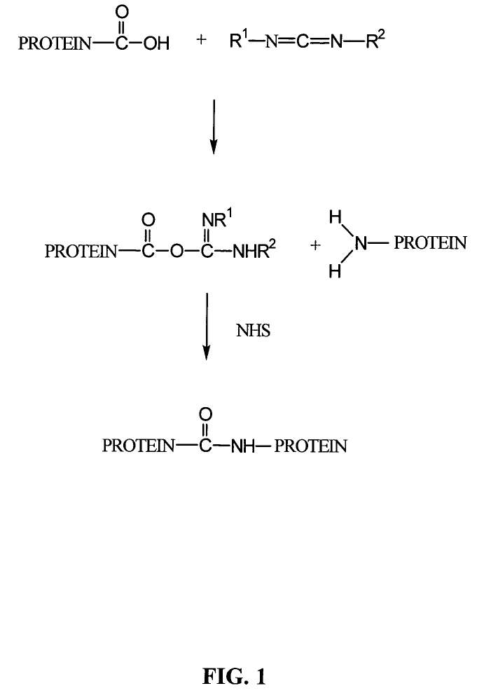

According to a further embodiment of the invention, a carbodiimide such as N-(3-dimethylaminopropyl)-N-ethylcarbodiimide hydrochloride (EDC) can be used to chemically cross-link the composition. FIG. 1 illustrates the formation of an amide crosslinked protein matrix using a carbodiimide. As shown in FIG. 1, a free carboxylic acid group on a first protein molecule reacts with the carbodiimide to form an O-acylisourea group. The carboxylic acid group can, for example, be on a glutamic or aspartic acid residue of a collagen molecule. The resulting O-acylisourea group can then react with an amine group on a second protein molecule to form the crosslink. The amine group can, for example, be on a hydroxy lysine residue of a collagen molecule.

Although crosslinks between collagen molecules are discussed above, crosslinks can also be formed between DBM and collagen. For example, carboxylic acid groups on the surface of the demineralized bone matrix can react with the carbodiimide and the resulting O-acylisourea group can then react with an amine group on a collagen molecule.

According to a further embodiment of the invention, the collagen matrix can be cross-linked with a carbodiimide (e.g., EDC) in the presence of N-hydroxysuccinimide (NHS). The addition of NHS during the crosslinking reaction can increase the crosslinking reaction rate thereby resulting in a collagen/DBM composition with a higher crosslink density relative to that of a composition formed without using NHS.

According to a further embodiment of the invention, the collagen matrix can be cross-linked with EDC under buffered or controlled pH conditions. Various crosslinking conditions are disclosed in International Publication No. WO 85/04413. Exemplary crosslinking conditions include, but are not limited to, a carbodiimide concentration of 10 to 300 mM, a reaction temperature of from 2 to 40° C., a pH of between 2 to 11, and a reaction time of about 1 to about 96 hours. Further exemplary reaction conditions include a carbodiimide concentration of 20 to 200 mM, a reaction temperature of from 10 to 35° C., a pH of between 3 and 9, and a reaction time of about 2 to 48 hours. Additional exemplary reaction conditions include a carbodiimide concentration of 50 to 150 mM, a reaction temperature of from 20 to 30° C., a pH of between 4 and 6.5, and a reaction time of 4 to 24 hours.

Although EDC is disclosed above, other carbodiimide crosslinking agents including, but not limited to, cyanamide can also be used according to an embodiment of the invention.

Growth factors, cells, plasticizers, and calcium or phosphate containing compounds can also be added to the osteoinductive composition according to an embodiment of the invention.

The chemical crosslinking method allows the amount of DBM added to the matrix and the material handling characteristics to be optimized without significantly affecting the osteoinductive capacity of the DBM. This crosslinking method allows for the production of a collagen/DBM composition that can maintain its shape when hydrated and that can regain its height following compression when hydrated.

The collagen/DBM composition according to an embodiment of the invention can be cut to various shapes and maintains its structure when rolled to fit into a variety of implant configurations. The composition can remain intact within the implant site for a 6 to 10 week time frame. This time frame, however, depends on implantation site and patient-to-patient variability. The collagen, being a natural component, allows for cellular attachment and migation and can be remodeled by the cells present in the defect site.

According to a further embodiment, the composition can be in the form of small collagen sponges. These sponges can be packed into a defect site alone or combined with allograft or autograft tissue for bone or soft tissue repair. The small collagen sponges can, for example, be in the shape of cubes or rectangular solids. The sponges can have dimensions of 2-10 mm. Further, the sponges can be ground to a finer size and combined with saline or another diluent to create a paste material. This paste can be injected or packed into a wound site for bone or soft tissue repair.

Further, the implantation of a composition comprising DBM and collagen according to an embodiment of the invention provides a composition having both osteoinductive and osteoconductive properties for the promotion of bone formation.

According to an exemplary embodiment of the invention, the collagen protein is in a porous scaffolding. The collagen matrix, for example, can be in the form of a porous or semi-porous sponge. Alternatively, the collagen matrix may be in the form of a membrane, a fiber-like structure, a powder, a fleece, particles or fibers. The porous scaffolding can provide an osteoconductive matrix for bone ingrowth.

The DBM can be in the form of particles of any size or shape. For example, DBM particles having an average diameter of up to 5 mm can be used according to one embodiment of the invention. According to a further embodiment of the invention, DBM particles having an average diameter of from 2 to 4 mm can be used. According to another embodiment of the invention, the DBM can be in the form of particles having an average diameter of 53 to 850 μm. Larger or smaller particles can also be used, however, depending on the desired properties of the composition. The DBM in the composition can also be in the form of blocks or strips.

The collagen source can be allogeneic or xenogeneic relative to the mammal receiving the implant. The source of the collagen may be from human or animal sources, or could be in a recombinant form expressed from a cell line or bacteria. The recombinant collagen may be from yeast or from any prokaryotic cell. The collagen may be extracted from tissue by any known method. The collagen protein can be any type of collagen.

The composition according to an embodiment of the invention can comprise any amount of demineralized bone matrix (DBM). The amount of DBM can be varied to achieve desired properties in the composition. According to one embodiment of the invention, the composition can comprise from 2 to 95 wt/% DBM based on the combined weight of DBM and collagen solids. According to a further embodiment of the invention, the composition can comprise from 55 to 85 wt/% DBM based on the combined weight of DBM and collagen solids.

The osteoinductive bone repair composition according to an embodiment of the invention can also include one or more growth factors. The one or more growth factors can be present within or on the collagen matrix. For example, cytokines or prostaglandins may be present within or on the porous or semi-porous collagen matrix or within or on the DBM particles. The growth factor may be of natural origin or recombinantly or otherwise produced using conventional methods. Such growth factors are also commercially available. Combinations of two or more growth factors may be applied to the osteoinductive compositions to further enhance the osteoinductive or biologic activity of the implants.

Examples of growth factors that may be used, include, but are not limited to: transforming growth factorβ (TGF-β), such as TGF-β1, TGF-β2, and TGF-β3; transforming growth factor-α (TGF-α); epidermal growth factor (EGF); insulin like growth factor-I or II; interleukin-I (IL-I); interferon; tumor necrosis factor; fibroblast growth factor (FGF); platelet derived growth factor (PDGF); nerve growth factor (NGF); and other molecules that exhibit growth factor or growth factor-like effects. According to one embodiment of the invention, the growth factor can be a soluble growth factor.

The growth factor may be incorporated into the collagen prior to formation of the collagen matrix. Alternatively, the growth factor may be adsorbed onto the collagen matrix in an aqueous or non-aqueous solution. For example, a solution comprising the growth factor may be infiltrated into the collagen matrix. According to a further embodiment, a solution comprising the growth factor may be infiltrated into the collagen matrix using vacuum infiltration.

The growth factor or factors can be delivered to the collagen demineralized bone matrix compositions in a liquid form. However, the growth factor can also be provided in a dry state prior to reconstitution and administration onto or into the collagen-demineralized bone matrix compositions. The growth factor present on or within the collagen matrix may reside within the void volume of the porous or semi-porous matrix. Growth factors contained within a controlled release carrier may also be incorporated into the collagen-demineralized bone matrix compositions.

Any known method of forming a porous collagen scaffolding can be used. For example, the DBM and collagen in the form of a slurry (e.g., an aqueous slurry) can be cast into the cavity of a mold having the desired shape and freeze dried to form the scaffolding. After the dried scaffolding is removed from the mold, the carbodiimide cross-linking agent can then be infiltrated into the pores of the composition and allowed to react with the collagen matrix and DBM to form the crosslinks.

Following is a description of non-limiting examples of reaction methods that can be used to form crosslinked collagen/DBM compositions.

Reaction Method 1

EDC at 10-300 mM concentration in water can be added to the porous collagen/DBM composition and allowed to react from 1-48 hours to cause collagen crosslinking.

Reaction Method 2

EDC at 10-300 mM concentration in MES buffer at pH 4.0-6.5 can be added to the porous collagen/DBM composition and allowed to react from 1-48 hours to cause collagen crosslinking.

Reaction Method 3

EDC at 10-300 mM concentration with NHS at an EDCINHS ratio of 1:2 to 2:5 (e.g., 1:2, 2:3, or 2:5) in water can be added to the porous collagen/DBM composition and allowed to react from 1-48 hours to cause collagen crosslinking.

Reaction Method 4

EDC at 10-300 mM concentration with NHS at an EDC/NHS ratio of 1:2 to 2:5 (e.g., 1:2, 2:3, or 2:5) in MES buffer at pH 4.0-6.5 added to the porous collagen/DBM composition and allowed to react from 1-48 hours to cause collagen crosslinking.

According to an exemplary embodiment of the invention, the chemically cross-linked collagen/DBM compositions can be used as a bone graft substitute (e.g., as a void filler). The chemically cross-linked collagen/DBM compositions can, for example, be implanted into a mammal (e.g., a human). According to one embodiment of the invention, the chemically cross-linked collagen/DBM composition can be implanted into the spine of a mammal. According to a further embodiment of the invention, the chemically cross-linked collagen/DBM composition can be implanted into an intervertebral space of a mammal.

Experimental

Collagen sponges were made from a 60 % DBM, 40 % collagen slurry. The collagen slurry and DBM particles were combined and blended to a uniform consistency. The mixture was poured into a mold, frozen, and freeze-dried. The dried sponges were exposed to the crosslinking solution at room temperature overnight. The crosslinking solution consisted of 100 mM EDC in water. Following the crosslinking, the sponges were rinsed 5 times with water. Sponges were frozen and then freeze dried. Sponges were then packaged in pouches and sterilized via E-beam irradiation.

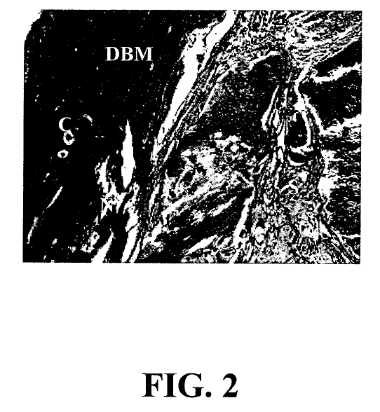

Sponges were then implanted into the athymic rat intramuscular pouch model (hind limb) for 4 weeks. Samples were then explanted, histological sections were prepared, and sections were stained with Hemotoxylin & Eosin. Images taken of the histological sections of the samples are shown in FIGS. 2-7.

FIG. 2 is an image of a section of a first sponge. The image was taken at 20× magnification. Sponge 1 comprised 80% DBM and 20% collagen. The sponge was made by combining DBM particles with a collagen slurry. The resulting mixture was poured into a mold, frozen and freeze dried into a sponge configuration. The sponge was exposed to a 100 mM EDC solution in water overnight. The resultant crosslinked sponge was rinsed with water several times, frozen and freeze dried. This final product was sterilized via E-beam irradiation at a dose of 25 kGy. Implantation samples were then cut to 3 mm cubes. These cubes were hydrated with a few drops of saline and implanted into the muscle pouch on the hind limb of athymic rats. The muscle pouch was sutured closed, and the animals were maintained under unrestricted conditions for 4 weeks. The animals were then sacrificed, and the sample removed with the surrounding muscle tissue. The explant was fixed in 10% neutral buffered formalin. Samples were processed through standard paraffin embedding techniques, sectioned and stained with Hematoxylin and Eosin. Sections were viewed under a standard light microscope using a 20× objective to analyze for osteogenic or chondrogenic activity.

In FIG. 2, the presence of chondrogenic activity (C) within a DBM particle (DBM) can be seen. A small area of new bone (N) can also be seen as can residual collagen sponge (S).

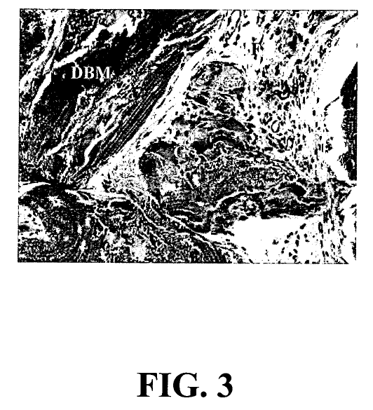

FIG. 3 is an image of a section of a second sponge (Sponge 2). The image shown in FIG. 3 was taken at 20× magnification. Sponge 2 comprised 80% DBM and 20% collagen. Sponge 2 was made by combining DBM particles with a collagen slurry. The resulting mixture was poured into a mold, frozen and freeze dried into a sponge configuration. The sponge was exposed to a 10 mM EDC solution in water overnight. The resultant crosslinked sponge was rinsed with water several times, frozen and freeze dried. The resulting product was sterilized via E-beam irradiation at a dose of 25 kGy. Implantation samples were cut to 3 mm cubes. These cubes were hydrated with a few drops of saline and implanted into the muscle pouch on the hind limb of athymic rats. The muscle pouch was sutured closed, and the animals were maintained under unrestricted conditions for 4 weeks. The animals were then sacrificed, and the sample removed with the surrounding muscle tissue. The explant was fixed in 10% neutral buffered formalin. Samples were processed through standard paraffin embedding techniques, sectioned and stained with Hematoxylin and Eosin. Sections were viewed under a standard light microscope using a 20× objective to analyze for osteogenic or chondrogenic activity.

In FIG. 3, the presence of fibrous tissue (F) and DBM particle (DBM) can be seen. Also, the presence of giant cells remodeling DBM (G) can be seen in FIG. 3.

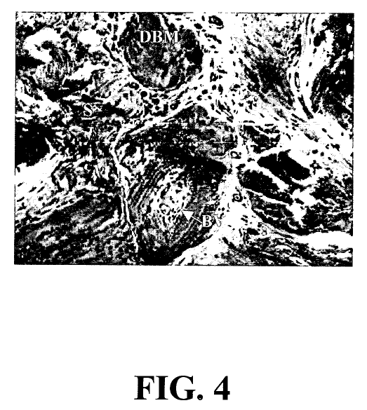

FIG. 4 is an image of another section of the second sponge (Sponge 2). This image was also taken at 20× magnification. In FIG. 4, the presence of a blood vessel (BV) within a DBM particle (DBM) can be seen. Residual collagen sponge (S) an also be seen in FIG. 4.

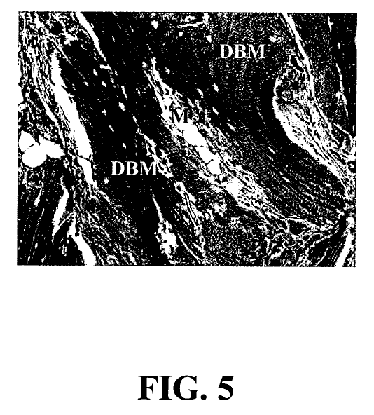

FIG. 5 is an image of a further section of the second sponge (Sponge 2). This image was also taken at 20× magnification. In FIG. 5, rudimentary marrow formation (C) can be seen between DBM particles (DBM).

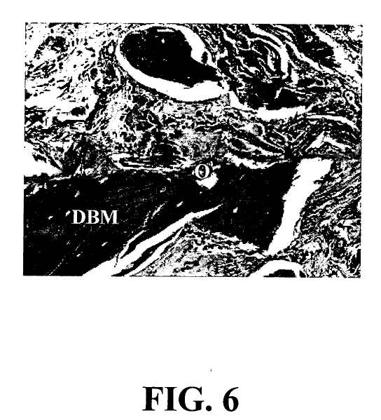

FIG. 6 is an image of a section of a third sponge. This image was also taken at 20× magnification. This sponge comprised 60% DBM and 40% collagen. Sponge 3 was made by combining DBM particles with a collagen slurry. The resulting mixture was then poured into a mold, frozen and freeze dried into a sponge configuration. The sponge was exposed to a 100 mM EDC solution in water overnight. The resultant crosslinked sponge was rinsed with water several times, frozen and freeze dried. This final product was sterilized via E-beam irradiation at a dose of 25 kGy. Implantation samples were cut to 3 mm cubes. These cubes were hydrated with a few drops of saline and implanted into the muscle pouch on the hind limb of athymic rats. The muscle pouch was sutured closed and the animals were maintained under unrestricted conditions for 4 weeks. The animals were then sacrificed, and the sample removed with the surrounding muscle tissue. The explant was fixed in 10% neutral buffered formalin. Samples were processed through standard paraffin embedding techniques, sectioned and stained with Hematoxylin and Eosin. Sections were viewed under a standard light microscope using a 20× objective to analyze for osteogenic or chondrogenic activity.

In FIG. 6, a demineralized bone matrix (DBM) particle lined by osteoblast-like cells (O) can be seen.

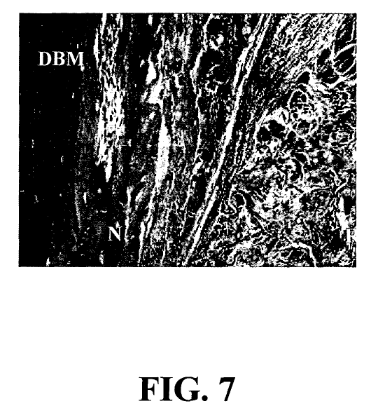

FIG. 7 is an image of a section of a fourth sponge. This image was also taken at 20× magnification. The sponge shown in FIG. 7 comprised 40% DBM and 60% collagen. The sponge was made by combining DBM particles with a collagen slurry. The resulting mixture was then poured into a mold, frozen and freeze dried into a sponge configuration. The sponge was exposed to a 100 mM EDC solution in water overnight. The resulting crosslinked sponge was rinsed with water several times, frozen and freeze dried. This final product was sterilized via E-beam irradiation at a dose of 25 kGy. Implantation samples were cut to 3 mm cubes. These cubes were hydrated with a few drops of saline, and implanted into the muscle pouch on the hind limb of athymic rats. The muscle pouch was sutured closed, and the animals were maintained under unrestricted conditions for 4 weeks. The animals were then sacrificed, and the sample removed with the surrounding muscle tissue. The explant was fixed in 10% neutral buffered formalin. Samples were processed through standard paraffin embedding techniques, sectioned and stained with Hematoxylin and Eosin. Sections were viewed under a standard light microscope using a 20× objective to analyze for osteogenic or chondrogenic activity.

In FIG. 7, demineralized bone matrix (DBM) with a small area of new bone (N) can be seen. Additionally, residual collagen sponge (R) can also be seen in FIG. 7.

The images of FIGS. 2 to 7 demonstrate that crosslinked collagen/DBM compositions as described herein can be used as implants to provide an osteoinductive and osteoconductive composition for the promotion of bone formation.

According to further embodiments of the invention, a composition is provided comprising demineralized bone matrix (DBM) and a collagen protein wherein the composition is chemically crosslinked with a compound selected from the group consisting of gluteraldehyde, formaldehyde, 1,4-butanediol diglycidyl ether, hydroxypyridinium, hydroxylysylpyridinium, and formalin.

A composition comprising demineralized bone matrix (DBM) and a collagen protein is also provided wherein the composition is crosslinked using irradiation (e.g., e-beam or gamma irradiation), light (e.g., ultraviolet light or other wavelengths of light using an appropriate initiator), or via photooxidation. When light is used for crosslinking, pulsed light may be used. The collagen matrix can also be crosslinked under dehydrothermal conditions or acidic conditions. For example, the composition can be crosslinked under dehydrothermal conditions by subjecting the composition to a vacuum at elevated temperature.

The composition may also be crosslinked using an enzymatic process. For example, the collagen may be crosslinked using lysyl oxidase or tissue transglutaminase. Lysyl oxidase is a metalloprotein which works by crosslinking collagen via oxidative deamination of the epsilon amino groups in lysine.

The collagen matrix can also be crosslinked by glycation (i.e., the nonenzymatic crosslinking of amine groups of collagen by reducing sugars, such as glucose and ribose) or glycosylation (i.e., the nonenzymatic attachment of glucose to collagen which results in a series of chemical reactions that result in the formation of irreversible cross-links between adjacent protein molecules). For example, the crosslinks may be pentosidine crosslinks (i.e., crosslinks resulting from the non-enzymatic glycation of lysine and arginine residues). Alternatively, the crosslinks in the collagen can be epsilon(gamma-glutamyl)lysine crosslinks.

The crosslinking may also be cellular driven. For example, crosslinking may result from culturing a non-crosslinked matrix in vivo to allow collagen crosslinking by cellular mechanisms.

The crosslinked collagen/DBM compositions can be implanted into a mammal to promote tissue formation. For example, the crosslinked collagen/DBM compositions can be implanted into a mammal to promote bone formation. Alternatively, the crosslinked collagen/DBM compositions can be implanted into a mammal to promote soft tissue formation. The crosslinked collagen/DBM compositions can be used in orthopaedic applications, in craniomaxillofacial applications, and for trauma injuries.

A spacer can be incorporated into the collagen/DBM compositions during crosslinking. Exemplary spacers include, but are not limited to, a polyoxyalkyleneamine (e.g., Jeffamine®, which is a registered trademark of Huntsman Corporation), a polyethylene glycol, or a polymeric spacer.

Vinyl pyrrolidinone and methyl methacrylate may also be incorporated into the crosslinked collagen/DBM compositions.

Bound or non-bound additives such as collagenase inhibitors, growth factors, antibodies, metalloproteinases, cell attachment fragment(s), or combinations thereof can also be incorporated into the crosslinked collagen DBM compositions. For example, one or more of these additives may be incorporated into the composition prior to or during crosslinking such that the additive becomes bound to the collagen or DBM.

While the foregoing specification teaches the principles of the present invention, with examples provided for the purpose of illustration, it will be appreciated by one skilled in the art from reading this disclosure that various changes in form and detail can be made without departing from the true scope of the invention.

1. A composition comprising:
demineralized bone matrix (DBM); and
a collagen protein;
wherein the composition is cross-linked.

2. The composition of claim 1, wherein the composition is chemically crosslinked with a carbodiimide crosslinking agent.

3. The composition of claim 2, wherein the carbodiimide crosslinking agent is N-(3-dimethylaminopropyl)-N-ethylcarbodiimide hydrochloride (EDC).

4. The composition of claim 2, wherein the composition is chemically cross-linked in the presence of N-hydroxysuccinimide (NHS).

5. The composition of claim 1, further comprising one or more growth factors.

6. The composition of claim 1, wherein the composition comprises from 2 to 95 wt/% DBM.

7. The composition of claim 1, wherein the composition comprises from 55 to 85 wt/% DBM.

8. The composition of claim 1, wherein the DBM comprises particles of the DBM dispersed in the collagen.

9. The composition of claim 1, wherein the collagen protein is in a porous scaffolding.

10. The composition of claim 9, wherein the DBM comprises particles of DBM dispersed in the porous scaffolding.

11. The composition of claim 8, wherein the DBM particles have an average particle size of up to 5 mm.

12. The composition of claim 8, wherein the DBM particles have an average particle size ranging from 53 to 850 μm.

13. The composition of claim 1, wherein the composition is chemically crosslinked with a compound selected from the group consisting of gluteraldehyde, formaldehyde, 1,4-butanediol diglycidyl ether, hydroxypyridinium, hydroxylysylpyridinium, and formalin.

14. The composition of claim 1, wherein the composition is crosslinked by irradiation.

15. The composition of claim 1, wherein the composition is crosslinked by photooxidation.

16. The composition of claim 1, wherein the composition is crosslinked via an enzymatic process.

17. The composition of claim 16, wherein the collagen protein is crosslinked via the action of tissue transglutaminase.

18. The composition of claim 16, wherein the composition is crosslinked with lysyl oxidase.

19. The composition of claim 1, wherein the composition is crosslinked by a dehydrothermal treatment.

20. The composition of claim 1, wherein the composition is crosslinked under acidic conditions.

21. The composition of claim 1, wherein the collagen protein is crosslinked using e-beam irradiation, gamma irradiation, or light.

22. The composition of claim 21, wherein the collagen protein is crosslinked using pulsed light.

23. The composition of claim 1, further comprising a spacer.

24. The composition of claim 23, wherein the spacer is a polyoxyalkyleneamine spacer or a polyethylene glycol spacer.

25. The composition of claim 1, wherein the composition further comprises vinyl pyrrolidinone or methyl methacrylate.

26. The composition of claim 1, further comprising an additive selected from the group consisting of collagenase inhibitors, growth factors, antibodies, metalloproteinases, cell attachment fragment(s), and combinations thereof.

27. The composition of claim 26, wherein the additive is bound to the collagen or DBM.

28. The composition of claim 26, wherein the additive is not bound to the collagen or DBM.

29. The composition of claim 1, wherein the composition is crosslinked by glycation or glycosylation.

30. The composition of claim 1, wherein the crosslinks are pentosidine crosslinks.

31. The composition of claim 1, wherein the crosslinks are epsilon(gamma-glutamyl)lysine crosslinks.

32. A method of making a composition comprising a collagen protein and demineralized bone matrix, the method comprising:
crosslinking the composition.

33. The method of claim 32, wherein the composition is chemically crosslinked with a carbodiimide crosslinking agent.

34. The method of claim 33, wherein the carbodiimide is N-(3-dimethylaminopropyl)-N-ethylcarbodiimide hydrochloride (EDC).

35. The method of claim 33, wherein the composition is chemically cross-linked in the presence of N-hydroxysuccinimide (NHS).

36. The method of claim 35, wherein the NHS is present at an EDC/NHS ratio of 1:2 to 2:5.

37. The method of claim 35, wherein the NHS is present at an EDC/NHS ratio of 1:2, 2:3 or 2:5.

38. The method of claim 32, further comprising dispersing particles of the demineralized bone matrix in a collagen slurry, casting the slurry into the cavity of a mold and freeze drying the cast slurry to form a porous scaffolding comprising the collagen protein and particles of the demineralized bone matrix.

39. The method of claim 38, wherein the slurry is an aqueous slurry.

40. The method of claim 38, wherein crosslinking comprises:
infiltrating a carbodiimide crosslinking agent into pores of the porous scaffolding; and
allowing the carbodiimide cross-linking agent to react with the collagen protein and/or the DBM to form cross-links.

41. The composition of claim 32, wherein the crosslinking results from culturing a non-crosslinked matrix in vivo to allow collagen crosslinking by cellular mechanisms.

42. A method of treatment comprising implanting a composition comprising demineralized bone matrix (DBM) and a collagen protein into a mammal, wherein the composition is crosslinked.

43. The method of claim 42, wherein the composition is chemically crosslinked with a carbodiimide crosslinking agent.

44. The method of claim 42, wherein the composition is implanted into the spine of the mammal.

45. The method of claim 42, wherein the composition is implanted into an intervertebral space of the mammal.

46. The method of claim 42, wherein the composition is implanted into the site of a trauma injury.

47. The method of claim 42, wherein the composition is implanted into a craniomaxillofacial cavity.

48. The method of claim 42, wherein the mammal is a human.

49. A composition comprising:
demineralized bone matrix (DBM); and
a collagen protein;
wherein the composition is cross-linked via an amide linkage.

50. The composition of claim 49, further comprising one or more growth factors.

51. The composition of claim 49, wherein the composition comprises from 2 to 95 wt/% DBM.

52. The composition of claim 49, wherein the composition comprises from 55 to 85 wt/% DBM.

53. The composition of claim 49, wherein the composition comprises particles of the DBM dispersed in the collagen protein.

54. The composition of claim 49, wherein the collagen protein is in a porous scaffolding.

55. The composition of claim 54, wherein the composition comprises particles of the DBM dispersed in the porous scaffolding.

56. The composition of claim 55, wherein the DBM particles have a particle size of up to 5 mm.

57. The composition of claim 55, wherein the DBM particles have a particle size of from 53 to 850 μm.
